(19) United States
(12) Patent Application Publication
(10) Pub. No.: US 2006/0030489 A1
(43) Pub. Date: Feb. 9, 2006

(54) Aquatic plant product and method for making growth-sustaining plant matrix

(76) Inventors: Donald Owen Northcott, Cornwall (CA); Mark O. Hamran, Tea, SD (US)

Correspondence Address: MARIO D. THERIAULT, 812 HWY. 101 NASONWORTH, FREDERICTON, NB, E3C 2B5, CA

(21) Appl. No.: 11/193,503

(22) Filed: Aug. 1, 2005

Related U.S. Application Data
(60) Provisional application No. 60/599,985, filed on Aug. 9, 2004.

(51) Int. Cl. A01N 39/02; A01H 9/00

(52) U.S. Cl. 504/323; 800/295

(57) ABSTRACT

The invention consists of an aquatic plant product for resale, having an extended shelf life, and a method for making the aquatic plant product. The aquatic plant product is made of a sealed container containing aquatic-plant-growth-sustaining medium therein and an aquatic plant planted in that aquatic-plant-growth-sustaining medium. The aquatic-plant-growth-sustaining medium contains a root-support material; at least one vitamin; sucrose; agar; distilled water; micro-nutrients containing iron; at least one hormone selected from a group consisting of auxin, naphthleneacetic acid, indole-3-butric acid, cytokine, 2-isopentyladenine, 6 Benzylaminopurine and any combination thereof.

This application claims the benefits of provisional application Ser. No. 60/599,985, filed on Aug. 9, 2004.

### FIELD OF THE INVENTION

This invention pertains to retail packages of aquatic plants, and more specifically, it pertains to sealed containers having growth-sustaining media therein providing extended shelf life for the plants therein.

### BACKGROUND OF THE INVENTION

It is the dream of every fish hobbyist to decorate their living space with attractive, plant-filled aquariums. However, it is expensive and labor-intensive for amateur hobbyists, or even professional breeders, to obtain and grow a variety of aquarium plants due to the fact that they require specialized equipment to propagate. Moreover, aquatic plant retailers experience significant product losses with aquatic plant products, as the plants currently available must be transplanted directly upon receipt of shipment from a provider or will perish. Thus, what is needed is a shelf-stable aquatic plant product.

### SUMMARY OF THE INVENTION

The present invention relates to an aquatic plant product characterized by an extended shelf life, which product comprises at least one aquatic plant and a stable growth-sustaining plant matrix, and to methods useful for generating same. For example, the present invention provides a sealed container, e.g., a test tube, containing therein an aquatic plant, for example, a tissue-culture propagated aquatic plant, in a sterile environment and in a growth sustaining plant matrix.

In one embodiment, the present invention provides nutrient rich, non-toxic, foliar support matrix conditions to sustain an aquatic plant in a sealed container for an extended period of time, for example, at least one year. An aquatic plant can be laboratory cultured under specific growth parameters and conditioned for entry into a container that provides the plant with sufficient nutrients and air to survive un-aided and without the addition of water or other normal plant requirements.

In one embodiment, the present invention provides a tissue culture medium formulated to sustain the aquatic plant of interest, for example, a tissue-culture propagated aquatic plant, in a contained, sterile environment for up to one year. The tissue culture medium is specific for the plant, and has precise nutritive components, e.g., hormones, fertilizers, amino acids, trace elements and minerals, such as fertilizer and trace minerals which aid in the growth and root formation, that sustain the individual plant.

For example, the medium according to the present invention comprises:
a) root-support material as described by inventors Murashige& Skoog, who are well known in this art;
b) at least one vitamin;
c) sucrose;
d) agar;
e) distilled water; and
f) at least one hormone selected from the list consisting of auxin, naphthleneacetic acid, indole-3-butric acid, cytokine, 2-isopentyladenine, 6 benzylaminopurine and any combination thereof.

Basically, the sealed container contains a plant-growth-supporting medium and a sterile aquatic plant. The plant-growth-supporting medium is water-based and by virtue of being sealed, establishes within the non-media portion of the container interior, the air space, a relative humidity that is substantially 100%. This 100% humidity level closely approximates conditions aquatic plants experience while submerged, this then creates the conditions required for successful growth of aquatic plants.

A sterile tissue-cultured aquatic plantlet when placed on a sterile plant-growth-supporting medium in a sealed vessel establishes an equilibrium in this environment and as such only grows at a rate supported by the factors that control the metabolic processes.

Plants complete two processes, photosynthesis and respiration. In the photosynthesis process, Oxygen is released from the plant as a metabolic process. In respiration, a plant absorbs Oxygen from the atmosphere and use it to drive metabolic processes. So the cycling of a limited supply of Oxygen within the closed system serves to regulate plant growth and in doing so it establishes the longevity of the plant in the closed system.

This brief summary has been provided so that the nature of the invention may be understood quickly. A more complete understanding of the invention can be obtained by reference to the following detailed description of the preferred embodiment thereof.

### DETAILED DESCRIPTION OF THE PREFERRED EMBODIMENT

I. Definitions

The following definitions are provided for specific terms that are used in the following written description:

As used in the specification and claims, the singular form “a,” “an” and “the” include plural references unless the context clearly dictates otherwise. For example, the term “a plant” includes a plurality of plants, e.g., aquatic plant species.

An “aquatic plant” refers to members of the kingdoms Plantae and Protista that are capable of survival and/or growth in an aquatic environment, e.g., under water or in a wetland environment.

“Container” includes any suitable, sealable device such as a tissue culture flask, tissue culture dish, test tube and the like. A container may be made of transparent material, such as glass, polystyrene and the like, or translucent material, such as polypropylene and the like.

The terms “medium” and “media” are used interchangeably with “substrate,” “matrix” and “formulation.”

The phrase “shelf-stable” refers to the capability of the product of the invention to sustain the plant being stored for an extended period of time, for example, for at least four years, at a temperature such as room temperature, e.g., 50° C.-85° C. In one embodiment of the invention, the shelf-stable aquatic plant product of the invention can be stored at room temperature for one to 4 years in the sterile container without having a deleterious effect on the ability of the aquatic plant to survive, e.g., grow and/or proliferate, once transplanted into an aquatic environment.

“Sterile” or “sterile environment” refers to the absence of prokaryotic, lower eukaryotes species, and/or pathogens.

II. Exemplary Plants of the Invention

The shelf-stable aquatic plant product of the invention includes a sterile, sealed container with at least one aquatic plant, the roots of which are in contact with a sustainable growth medium that is tailored to meet the nutritive needs of the plant(s) of interest.

Any aquatic and/or wetland plants can be used with the method of the present invention, such as any emergent, floating-leaved, submersed, and freely floating aquatic plant. Aquatic macrophytes, by definition, are the macroscopic (large enough to be observed by the naked eye) forms of aquatic and wetland plants found in water bodies. The term aquatic macrophytes refers to a diverse group of aquatic plants and encompasses flowering vascular plants, mosses, ferns, and macroalgae.

Emergent macrophytes (plants that are rooted in substrate with the tops of the plant extending into the air) grow on periodically inundated or submersed soils. Most emergent macrophytes are perennials (plants or plant parts living for greater than one year). They are typically rooted in the bottom, have their base portions submersed in water, and have their tops elevated into the air.

Emergent plants have to be strongly rooted and much energy is put into producing a strong structure to withstand the wind and waves in the shallow water zone.

Common emergent macrophytes include plants such as reeds (Phragmites spp.), bulrushes (Scirpus spp.), cattails (Typha spp.), and spikerushes (Eleocharis spp.). Some emergents, wild rice (Zizania spp.) for example, have submersed or floating leaves before mature aerial leaves form.

Floating-leaved macrophytes (plants that are rooted to the lake bottom with leaves that float on the surface of the water) generally occur in areas of a lake that do not occasionally dry out. Common representatives include waterlilies (Nymphaea spp.), spatterdock (Nuphar spp.), and watershield (Brasenia spp.). Floating leaves are attached to roots or rhizomes with a flexible, tough stem (actually in many cases a leaf stalk). Some floating-leaved macrophytes, like Nuphar spp., can exist in a submersed form for a considerable time. Many floating-leaved species form large colonies from spreading underground rhizomes.

Floating leaves live in two extremely different habitats, water on the bottom, air on top. A thick, waxy coating protects the top of the leaf from the aerial environment. Floating leaves can be ravaged by wind and waves so these plants are usually found in protected areas.

Submersed macrophytes (plants that grow completely submersed under the water) are a diverse group that includes quillworts (Isoetes spp.), mosses (Fontinalis spp.), muskgrasses (Chara spp.), stoneworts (Nitella spp.) and numerous vascular plants. Many submersed plants, such as widgeon-grass (Ruppia maritima), various pondweeds (Potamogeton spp.), and tape-grass (Vallisneria spp.), are native to the United States.

Submersed species face special problems obtaining light for photosynthesis and they must obtain carbon dioxide from the water where it is much less available than it is in air. Submersed species invest much less energy into structural support because they are supported by water and water accounts for about 95% of the weight of these submersed species.

Free floating macrophytes (plants that typically float on or just under the water surface with their roots in the water and not in sediment) are a diverse group of aquatic plants. Small free floating plants include duckweeds (Lemna spp.), mosquito fern (Azolla caroliniana) and water fern (Salvinia spp.). Larger surface floating plants include water hyacinth (Eichhornia crassipes), frog's bit (Limnobium spongia).

Free floating species are entirely dependent on the water for their nutrient supply. In fact, some (e.g., water hyacinth) have been used in wastewater treatment to remove excess nutrients.

Thus, the shelf-stable aquatic plant product of the invention includes a sterile, sealed container with one aquatic plant or a plurality of aquatic plants, for example, a combination of one or more aquatic plant species. For example, plants suitable for aquarium growth include, but are not limited to aluminum plant (Pilea cadierei), ambulia (Limnophila indica), anacharis (Egeria densa), bacopa (Bacopa caroliniana), cabomba, green (Cabomba caroliniana), cabomba, purple (Cabomba pulcherrima), cardamine (Cardamine lyrata), creeping charlie (Micromeria brownei), glossostigma (Glossostigma elatinoides), hornwort (Ceratophyllum demersum), hygrophila, scarlet (Alternanthera sessilis), hygrophila, tropic sunset (Hygrophila polysperma), java moss (Vesicularia dubyana)(large clump), lloydiella (Lysimachia nummularia), lloydiella, golden (Lysimachia nummularia v. ‘aurea’), ludwigia, broad leaf (Ludwigia repens), ludwigia, narrow leaf (Ludwigia palustris), ludwigia, needle leaf (Ludwigia arcuata), myrio, filigree (Myriophyllum simulans), myrio, green (Myriophyllum pinnatum), myrio, red (Myriophyllum heterophyllum), temple, narrow leaf (Hygrophila corymbosa), barteri, barteri (Anubias barteri v. ‘barteri’), barteri, round leaf (Anubias barteri v. ‘round leaf’), coffeefolia (Anubias barteri v. ‘coffeefolia’), congensis (Anubias ‘congensis’), frazeri (Anubids ‘frazeri’), gigantea (Anubias gigantea), gracilis (Anubias gracilis), hastifolia (Anubias hastifolia), lanceolata (Anubias lanceolata), nana (Anubias barteri v. ‘nana’), capuronii (Aponogeton capuronii), natans (Aponogeton natans), rigidifolius (Aponogeton rigidifolius), balansae (Cryptocoryne crispatula), blassii (Cryptocoryne blassii), ciliata (Cryptocoryne ciliata), retrospiralis (Cryptocoryne retrospiralis), spiralis (Cryptocoryne spiralis), amazon, compacta (Echinodorus bleheri ‘compacta’), chain, broad leaf (Echinodorus quadricostatus), chain, narrow leaf (Echinodorus tenellus), hormanii, red (Echinodorus hormanii), indian red (Echinodorus ‘indian red ’), jade (Echinodorus hormanii), melon (Echinodorus osiris), melon, red (Echinodorus barthii), radican (Echinodorus cordifolius), radican, marble queen (Echinodorus cordifolius), rose (Echinodorus ‘rose’), rosette (Echinodorus parviflorus v. ‘tropica’), ruffle (Echinodorus martii), uruguayensis (Echinodorus uruguayensis), chilensis (Sagittaria platyphylla), subulata (Sagittaria subulata), subulata, dwarf (Sagittaria subulata), contortion (Vallisneria asiatica), corkscrew (Vallisneria americana), italian (Vallisneria spiralis), jungle (Vallisneria americana), barclaya (Barclaya longifolia), bolbitis (Bolbitis heudelotii), bolbitis (Bolbitis heteroclita), crinum natans (Crinum natans), crinum, “onion plant” (crinum thaianum), crinum, “onion plant”, dwarf (Zephyranthes candida), duckweed (Lemna minor), dwarf lily (Nymphaea stellata), java fern (Microsorium pteropus), java fern, lace (Microsorium pteropus v. ‘tropica’), micro sword (Lilaeopsis novae-zelandiae), tiger lotus, green (Nymphaea zenkeri), tiger lotus, red (Nymphaea zenkeri), water sprite (Ceratopteris thalictroides)

In one embodiment, the shelf-stable, sterile container of the invention includes at least one baby tears (Hemianthus micranthemoides), diandra (Didiplis diandra), heteranthera (Heteranthera zosterifolia), hygrophila (Hygrophila polysperma), ludwigia peruensis (Ludwigia peruensis), mayaca (Mayaca fluviatilis), mexican oak leaf (Shinnersia rivularis), moneywort (Bacopa monnieri), pennywort, brazilian (Hydrocotyle leucocephala), rotala indica (Rotala indica), rotala magenta (Rotala macrandra v. ‘narrow leaf’), rotala wallichii (Rotala wallichii), telanthera (Alternanthera reineckii), temple (Hygrophila corymbosa), wisteria (Hygrophila difformis), boivinianus (Aponogeton boivinianus), crispus (Aponogeton crispus), longiplumulosus (Aponogeton longiplumulosus), madagascar lace (Aponogeton madagascariensis), ulvaceus (Aponogeton ulvaceus), undulatas (Aponogeton undulatas), wendtii, green (Cryptocoryne wendtii), wendtii, red (Cryptocoryne wendtii), amazon (Echinodorus bleheri), brazil (Spathiphyllum tasson), oriental (Echinodorus ‘oriental ’), ozelot (Echinodorus ‘ozelot’), red rubin (Echinodorus ‘rubin’), banana plant (Nymphoides aquatica), crinum calamistratum (Crinum calamistratum), or any combination thereof.

Other aquatic plants which can be packaged and grown or preserved alive in the container and the media according to the present invention includes: Aeschynomne fluitans; Aglaonema ‘Minima™’; Aglaonema ‘Silver Queen™’; Aglaonema simplex; Alternanthera ‘Red Round™’; Alternanthera ficiodea bettzickiana ‘Red™’; Alternanthera ficodea bettzickiana ‘Green™’; Alternanthera ficoidea bettzickiana ‘Aurea™’; Altemanthera lilacina; Alternanthera lilacina ‘Variegated™’; Altemanthera ocipus; Ammania ‘Bonsai™’; Ammania gracilis; Anubias ‘Gabon™’; Anubias barteri var barteri ‘Broad Leaf™’; Anubias barteri var. barteri; Anubias barteri var. barteri ‘Marble™’; Anubias barteri var. barteri ‘Oriental Green™’; Anubias barteri var. barteri ‘Sharp Wavy Leaf™’; Anubias barteri var. barteri ‘Variegated™’; Anubias barteri var. nana ‘Eyes™’; Anubias barteri var. nana ‘Golden™’; Anubias barteri var. nana ‘Narrow Leaf™’; Anubias barteri var. nana ‘Stardust™’; Anubias barteri var. nana ‘Wrinkled Leaf™’; Anubias barteri var. nana ‘Yellow Heart™’; Apongeton henkalianus; Apongeton stachysporus; Bacopa australis; Bacopa lanigera; Bacopa myriophylloides; Blyxa alternifolius; Blyxa aubertii; Blyxa japonica; Bolbitis heteroclitia; Cabomba aquatica; Cabomba palaeformis; Cabomba piaauhyensis; Caladium ‘Freda Hemple™’; Caladium humboldtii ‘White™’; Ceratopteris siliquosa; Chloroophytum bichetii; Cordyline; Cordyline ‘Compacta™’; Cordyline ‘Red Edge Special™’; Cordyline ‘Red Edge™’; Crassula helmsii; Crinum aquatica; Cryptocoryne affinis; Cryptocoryne albida; Cryptocoryne becketti; Cryptocoryne blassii; Cryptocoryne lingua; Cryptocoryne longicula; Cryptocoryne lucens; Cryptocoryne parva; Cryptocoryne petchii; Cryptocoryne pontederiifolia; Cryptocoryne pygmaea; Cryptocoryne tonkinensis; Cryptocoryne tropica; Cryptocoryne usteriana; Cryptocoryne willisii; Cyperus alternifilius; Cyperus haspens; Cyperus helferi; Dieffenbachia ‘Exotica Variegatus™’; Dieffenbachia bausei; Dieffenbachia marianna; Dieffenbachia picta; Echinodorus ‘Apart™’; Echinodorus ‘Long Leaf™’; Echinodorus ‘Narrow Leaf Red Rubin™’; Echinodorus ‘Python™’; Echinodorus ‘Red Flame™’; Echinodorus amazonicus; Echinodorus angustifolia; Echinodorus argentinesis; Echinodorus cordifolius; Echinodorus gabrieli; Echinodorus grandifolius; Echinodorus harbich; Echinodorus horemanii ‘Green™’; Echinodorus horemanii ‘Red™’; Echinodorus latifolius; Echinodorus major; Echinodorus muricatus; Echinodorus muricatus ‘Green™’; Echinodorus scaber; Echinodorus schlueteri; Echinodorus schlueteri ‘Leopard™’; Eichhornia crassipes; Eichhornia diversifolia; Eleocharis parvulus; Eleocharis vivipara; Eriocaulon cineteum; Eustrlis setllata; Fittonia argyronanta; Gymnocoronis spilanthoides; Gymnocoronis spilanthoides variegated; Hemianthus callitrichoides; Hemigraphis c. ‘Exotica™’; Hemigraphis colorata; Hemigraphis repanda; Heteranthera dubia; Hottonia palustris; Hydrocharis dubia; Hydrocharis nymphaeoides; Hydrocotyle leuncocephala; Hydrocotyle sibthorpiodes; Hydrocotyle verticillata; Hydrotriche hottoniiflora; Hygrophila ‘Red™’; Hygrophila augustifolia; Hygrophila lacustris; Hygrophila salicifolia; Hygrophila salicifolia ‘Long Form™’; Hygrorhyza aristata; Lagarosiphon madgascarensis; Lagenandra thwaitesii; Lilaeopsis ‘Mauritius™’; Lilaeopsis brasilliensis; Limnobium laevigatum; Limnophila ‘Gigantea™’; Limnophila ‘Taiwain™’; Limnophila aquatica; Limnophila aromatica; Limnophila huppuroides; Limnophila rotundifolia; Limnophila rotundifolia ‘Variegated’; Lobelia cardinalis ‘Small Form™’; Ludwigia ‘Cuba™’; Ludwigia arcuata; Ludwigia brevipes; Ludwigia glandulosa; Ludwigia inclinata; Ludwigia ovalis; Ludwigia palustris ‘Green™’; Ludwigia sedioides; Lysimachia nummularia; Lysimachia nummularia ‘Aurea™’; Marsilea angustifolia; Marsileacrenata; Mayaca sellowiniana; Micrathemum micranthemoides; Microcarpaea minima; Microsorium pterpus ‘Narrow Leaf™’; Monosolenium tenerum; Myriophyllum dicrocum; Myriophyllum elatinoides; Myriophyllum hippuroides; Myriophyllum matogrossense ‘Green Form™’; Myriophyllum procerpinacoides; Myriophyllum propinqum; Myriophyllum scabratum; Myriophyllum tuberculatum; Najas indica; Nesaes; Nesaes pedicellata; Nuphar japonicum ‘Spatterdock™’; Nymphaea micrantha; Nymphaea rubra; Nymphaea stellata; Nymphoides; Nymphoides indica; Ophiopogon jaburan; Ophiopogon jaburan variegatus; Ophiopogon japonica; Ophiopogon japonica ‘Kyoto™’; Ottelia alismoides; Ottelia ovalifolia; Peliosanthes; Physostegia purpurea; Pista stratiotes; Pogostemon stellata; Pogostemon stellata ‘Broad Leaf™’; Polygonum; Polygonum amphibium; Polygonum pedunculare; Potamageton ‘Malainus™’; Potamageton crispus; Potamageton gayi; Potamageton octandra; Potamageton perfoliatus; Proserpinaca palustris; Ranalisma rostrata; Rorippa aquatica; Rotala ‘Pearl™’; Rotala macrandra ‘Green™’; Rotala macrandra ‘variegated™’; Rotala najean; Rotala rotundifolia ‘Green™’; Rotalawallichii ‘Long Leaf™’; Saururus cernuus; Selaginella wildenowii; Syngoium ‘White Butterfly™’; Syngoium podophyllum; Syngoium podophyllum ‘Red Knight™’; Tonina fluviatilis; Trapa natans; Trichocoronis rivularis; Trichocoronis rivularis variegatus; Trichomanes javanicum ‘Aqua Fern™’; Ultricularia; Vallisneria americana var. americana ‘Rubra™’; Vallisneria americana var. biwaensis; Vallisneria americana var. natans; Vallisneria neotropicalis; Vallisneria rubra ‘Serpanta™’; Vallisneria spiralis ‘Leopard™’; Vallisneria spiralis ‘Red™’; Vesicularia ferriei ‘Weeping Moss™’; Vesicularia montagnei ‘Christmas Moss™’.

III. Methods of the Present Invention

Plant tissue culture is used for plant propagation. It operates on the principle of growing disease-free plant tissues under sterile conditions in artificial plant growth medium. Briefly, a superior plant is identified, checked and verified to have all the growth characteristics particular to the variety. These plants are designated to be the mother plants or the donor of plant tissues to start the tissue culture process. The mother plant should be healthy and free from all micro-organisms and a laboratory disease testing program followed to verify freedom from bacteria, viruses and other disease organisms.

A growing point or meristem is removed from a plant and placed into culturing flasks containing appropriate nutrient media and growth promoting conditions. The plant tissues grow on a nutrient rich culture medium in a sterile environment. Bacteria and fungi are excluded from the mother plant, it is also necessary to exclude these organisms from the growth of the plant tissue therefore, all manipulations are carried out under sterile conditions. At predetermined stages, the growing plants are divided under sterile conditions and in successful culture, these cells divide, multiply and differentiate into thousands of plantlets having the same characteristics as the mother plant. As the desired number of plantlets has been reached, the plantlets can be transferred to pots or greenhouse conditions to continue the growth process. These plantlets are uniform and with proper care will grow and produce a crop of identical healthy plants.

The tissue culture propagated aquatic plant(s) is then deposited into a sterile container, such as a plastic tube, under sterile conditions. The roots of the plant(s) are placed in contact with the substrate of the invention that has been formulated according to the individual needs of the plant(s) to be stored. Then, the container is sealed, and can be stored for an extended period of time, for example, on the shelf of a commercial vendor of plant material.

After being sealed in the sterile container, the aquatic plant can be placed, e.g., transplanted, into an aquatic environment.

IV. Plant Matrices of the Present Invention

As discussed herein, the sustained growth matrices of the invention are formulated according to the individual needs of an aquatic plant of interest. In one embodiment of the invention, a sustainable growth matrix is provided that contains a source of nutrients, such as a balance of macronutrients and minor or trace nutrients. Macronutrients can include but are not limited to oxygen (O₂), CO₂, nitrogen (N), phosphorus (P), sulfur (S), calcium (Ca), magnesium (Mg) and potassium (K). Micronutrients include but are not limited to iron (Fe), manganese (Mn), copper (Cu), zinc (Zn), molybdenum (Mo), cobalt (Co), and boron (B). In addition, the matrix can contain organic components such as adenine sulfate, activated charcoal, adenine hemisulfate, agar, 6-Benzylaminopurine (BA), D-biotin, 6 gamma-gamma-Dimethylallylaminopurine, glycine, indole-3-acetic acid, indole-3-butyric acid, kinetin, MES, myo-Inositol, alpha-Naphthaleneacetic acid, nictotinic acid, peptone, pyridoxine, sucrose, and/or thiamine.

The matrix of the invention also can contain a hormone, such as abscisic acid, an auxin, naphthaleneacetic acid (NAA), indole-3-butric acid (IBA), a cytokinin, a gibberellin, 2-isopentyladenine (2-IP), 6-benzylaminopurine (BAP), or any combination thereof.

The invention will now be illustrated by the following non-limiting example.

An aquatic plant-support medium prepared according to the following formula:

1. Root-support material as described by inventors Murashige & Skoog for example, with vitamins;
2. Sucrose;
3. Agar;
4. Distilled water;
5. One or more of the following hormones: auxin, NAA (Naphthleneacetic Acid), IBA (Indole-3-Butric Acid), cytokine, 2-iP (2-isopentyladenine) and BAP(6 Benzylaminopurine).

More particularly, the following ingredients of a preferred medium and their relative quantities are as follows, expressed in gram(s) per liter of water:

A preferred aquatic plant inorganic salt stock contains

| MgSO₄.7H₂O | 2.0 grams |
|---|---|
| MnSO₄.H₂O | 0.86 |
| ZnSO₄.7H₂O | 0.46 |
| CuSO₄.5H₂O | 0.001 |
| CaCl₂.2H₂O | 0.22 |
| KI | 0.04 |
| CoCl₂.6H₂O | 0.001 |
| KH₂PO₄ | 1.7 |
| H₃BO₃ | 0.32 |
| Na₂MoO₄.2H₂O | 0.01 |

A preferred iron stock preparation includes:

| | |
|---|---|
| Na₂EDTA | 1.5 grams |
| FeSO₄.7H₂O | 1.48 grams |

Preferred plant growth regulating hormones comprise: IAA, IBA, NAA, 2,4, D, BAP, Kinetin™, 2iP, Zeaitin™

While one embodiment of this invention has been described herein above, it will be evident to those skilled in the art that changes and modifications may be made therein without departing from the essence of this invention, as set forth in the appended claims.

What is claimed is:

1. A method for making a shelf-stable aquatic plant product comprising the steps of;
propagating a first aquatic plant by tissue-culture;
providing a sterile container with aquatic-plant-growth-sustaining medium therein; said medium containing root-support material; at least one vitamin; sucrose; agar; distilled water; micro-nutrients containing iron; at least one hormone selected from a group consisting of auxin, naphthleneacetic acid, indole-3-butric acid, cytokine, 2-isopentyladenine, 6 Benzylaminopurine and any combination thereof;
planting said aquatic plant in said medium, and
sealing the container under sterile conditions.

2. A shelf-stable aquatic plant product comprising:
a sealed container containing aquatic-plant-growth-sustaining medium therein; said aquatic-plant-growth-sustaining medium containing root-support material; at least one vitamin; sucrose; agar; distilled water; micro-nutrients containing iron; at least one hormone selected from a group consisting of auxin, naphthleneacetic acid, indole-3-butric acid, cytokine, 2-isopentyladenine, 6 Benzylaminopurine and any combination thereof; and
an aquatic plant planted in said aquatic-plant-growth-sustaining medium.

3. The shelf-stable aquatic plant product as claimed in claim 2, wherein said container comprises a test tube.

4. The shelf-stable aquatic product as claimed in claim 3, wherein said tube is transparent.

5. The shelf-stable aquatic plant product as claimed in claim 2, wherein said aquatic plant is a plant obtained by tissue-culture.

6. A shelf-stable aquatic plant product comprising:
a sealed container containing water-based, aquatic-plant-growth-sustaining medium therein;
an air space inside said container;
means for creating a 100% relative humidity in said air space; and
an aquatic plant in said container, planted in said aquatic-plant-growth-sustaining medium.

7. The shelf-stable aquatic plant product as claimed in claim 6, wherein said aquatic plant is a sterile tissue-cultured plantlet.

8. The shelf-stable aquatic plant product as claimed in claim 6, further including means for regulate a growth of said aquatic plant.

9. The shelf-stable aquatic plant product as claimed in claim 8 wherein said means for regulate a growth of said aquatic plant includes an Oxygen content of said air space.

10. The shelf-stable aquatic plant product as claimed in claim 6, wherein said medium further includes micro-nutrients containing iron.

11. The shelf-stable aquatic plant product as claimed in claim 6, wherein said container is made of a transparent material.

12. The shelf-stable aquatic plant product as claimed in claim 10, wherein said medium further includes an inorganic salt stock containing MgSO₄.7H₂O; MnSO₄.H₂O; ZnSO₄.7H₂O; CuSO₄.5H₂O; CaCl₂.2H₂O; KI; CoCl₂.6H₂O; KH₂PO₄; H₃BO₃; and Na₂MoO₄.2H₂O.

13. The shelf-stable aquatic plant product as claimed in claim 12, wherein said iron is comprised in a compound containing Na₂EDTA and FeSO₄.7H₂O.

14. The shelf-stable plant product as claimed in claim 13, wherein said medium further comprises plant nutrients selected from sucrose, Mannitol™, Inositol™, B1, B5 and Nicotinic Acid.

15. The shelf-stable plant product as claimed in claim 14, wherein said medium further comprises plant-growth regulating hormones selected from IAA, IBA, NAA, 2,4, D, BAP, Kinetin™, 2iP, and Zeaitin™
